(19) United States
(12) Patent Application Publication
(10) Pub. No.: US 2019/0282104 A1
(43) Pub. Date: Sep. 19, 2019

(54) BIOLOGICAL MEASUREMENT DEVICE AND BIOLOGICAL MEASUREMENT METHOD

(71) Applicants: Kabushiki Kaisha Toshiba, Tokyo (JP); Toshiba Electronic Devices & Storage Corporation, Tokyo (JP)

(72) Inventors: Ken Kawakami, Kawasaki Kanagawa (JP); Akira Iguchi, Yokohama Kanagawa (JP); Takashi Fujinami, Yokohama Kanagawa (JP)

(21) Appl. No.: 16/128,899

(22) Filed: Sep. 12, 2018

(30) Foreign Application Priority Data
Mar. 13, 2018 (JP) 2018-045197

(51) Int. Cl. A61B 5/021; A61B 5/024

(52) U.S. Cl. CPC A61B 5/02108; A61B 5/02405; A61B 5/02141

(57) ABSTRACT

A biological measurement device according to an embodiment includes a measurer, an evaluation value acquirer, and an output unit. The evaluation value acquirer measures a pulse wave. The evaluation value acquirer acquires, on the basis of a first index value based on an amplitude value of a speed pulse wave obtained by time-differentiating the pulse wave and a second index value correlating with a reference level of the pulse wave at each heartbeat, an evaluation value indicating heat radiation activity. The output unit configured to output information corresponding to the evaluation value.

### CROSS-REFERENCE TO RELATED APPLICATIONS

This application is based upon and claims the benefit of priority from Japanese Patent Application No. 2018-045197, filed on Mar. 13, 2018; the entire contents of which are incorporated herein by reference.

### FIELD

The present invention relates to a biological measurement device and a biological measurement method.

### BACKGROUND

In recent years, researches concerning a relation between peripheral circulation resistance of a human body and heat radiation activity of the human body due to the peripheral circulation resistance have been in progress. Developments of a wearable device that is worn on a forearm of a person to be measured and displays information such as a dehydration state have also been in progress.

However, in general, information concerning the peripheral circulation resistance of the human body is measured by a device of a Doppler measurement system or the like in which ultrasound or a laser beam is used. Therefore, it is difficult to realize a wearable device that acquires these kinds of information.

### BRIEF DESCRIPTION OF THE DRAWINGS

FIG. 1 is an exterior schematic view of a biological measurement device according to an embodiment;

FIG. 2 is a block diagram showing the configuration of the biological measurement device;

FIG. 3 is a detailed configuration diagram of a measurer;

FIG. 4 is a conceptual diagram showing a state of measurement of the measurer;

FIG. 5 is a diagram showing an example of a volume pulse wave measured by the measurer;

FIG. 6 is a diagram showing an example of a volume pulse wave at the time when heat stress is given to a person to be measured;

FIG. 7 is a diagram showing a relation between a volume pulse wave and a speed pulse wave for one heartbeat;

FIG. 8 is a diagram showing a relation between the amplitude of the speed pulse wave and an average blood flow value measured by a Doppler blood flow meter;

FIGS. 9A to 9D are diagrams showing examples of evaluation values;

FIG. 10 is a diagram showing a relation between an evaluation value and peripheral circulation resistance measured by the Doppler blood flow meter;

FIG. 11 is a diagram showing a radius vector (θ) at the time when the ground is set as an initial line and a fingertip is set as the origin of the initial line;

FIG. 12 is a diagram for explaining correction processing by a second acquirer;

FIG. 13 is a diagram showing a relation between evaluation values 1 and 2 and a body temperature and an average blood flow rate;

FIGS. 14A to 14E are diagrams showing a relation between evaluation values 2 and 3 and a body temperature and an HRV analysis at the time when a hot heat load is given;

FIGS. 15A to 15E are diagrams showing a relation between the evaluation values 2 and 3 and the body temperature and the HRV analysis on a measurement date different from a measurement date in FIGS. 14A to 14E;

FIGS. 16A to 16E are diagrams showing a relation between the evaluation values 2 and 3 and the HRV analysis at the time when the hot heat load is not given; and

FIG. 17 is a flowchart showing a processing example in which the evaluation values 1 and 2 are used.

### DETAILED DESCRIPTION

An embodiment of the present invention is explained below with reference to the drawings. Note that, in the drawings attached to this specification, for convenience of illustration and easiness of understanding, scales, aspect ratios, and the like are changed from actual ones and exaggerated.

### Embodiment

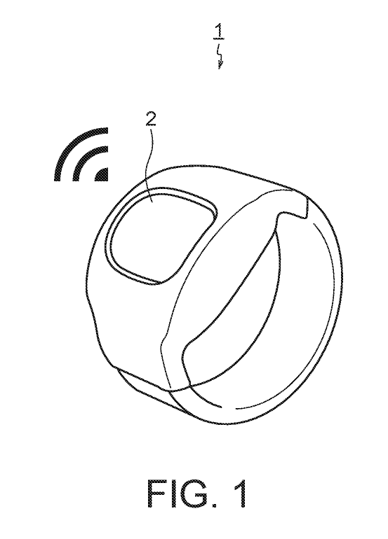

FIG. 1 is an exterior schematic view of a biological measurement device 1 according to an embodiment. The biological measurement device 1 shown in FIG. 1 is of a wristwatch type and can be worn on, for example, a forearm of a person to be measured. For example, the biological measurement device 1 is configured to be capable of presenting, on a display 2, information concerning an internal state of the person to be measured. In this way, the person to be measured can recognize the internal state during exercise and during outdoor work, for example.

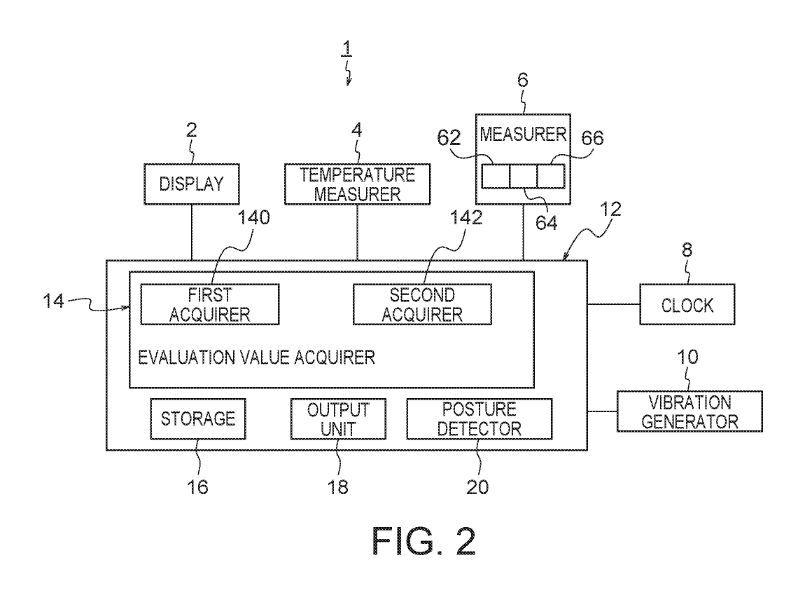

FIG. 2 is a block diagram showing the configuration of the biological measurement device 1. The biological measurement device 1 shown in FIG. 2 includes a display 2, a body temperature measurer 4, a measurer 6, a clock 8, a vibration generator 10, and a biological-information processing device 12.

The display 2 is, for example, a liquid crystal monitor and presents information input from the temperature measurer 4, the clock 8, the biological-information processing device 12, and the like. The temperature measurer 4 includes a temperature sensor and measures the temperature of the person to be measured. The temperature measurer 4 is capable of causing the display 2 to display the measured temperature and causing the vibration generator 10 to communicate the measured temperature as vibration.

The measurer 6 optically measures a change in a pulse wave of the person to be measured. The measurer 6 includes a light emitter 62, a light receiver 64, and a pulse wave generator 66. The clock 8 is a general clock and causes the display 2 to display time and the like. The vibration generator 10 transmits information input from the body temperature measurer 4, the clock 8, the biological-information processing device 12, and the like as vibration. The biological-information processing device 12 processes, for example, information concerning a volume pulse wave and obtains biological information of the person to be measured.

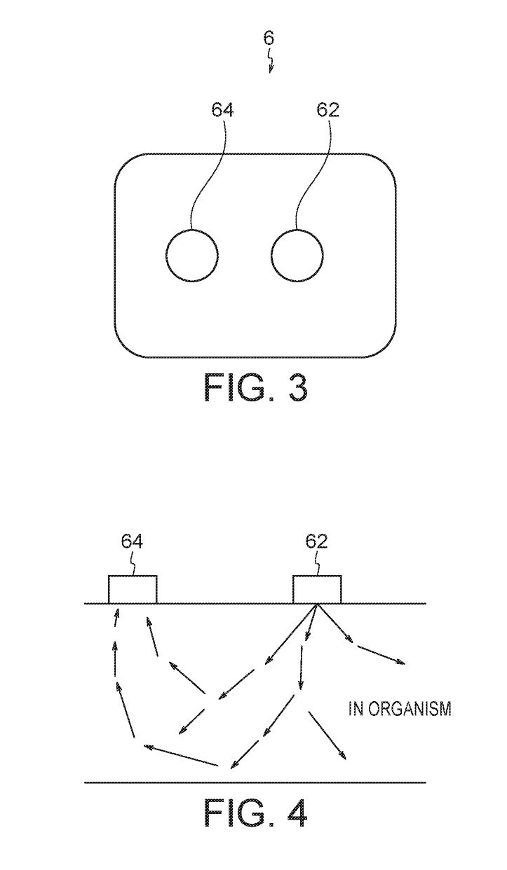

A detailed configuration of the measurer 6 is explained with reference to FIGS. 2 and 3. FIG. 3 is a configuration diagram of the measurer 6. The measurer 6 shown in FIG. 3 is, for example, a reflection type and is disposed on the rear side of the biological measurement device 1, that is, a side in contact with the skin of the person to be measured. Note that, although the measurer 6 according to this embodiment is the reflection type, the measurer 6 is not limited to this and may be, for example, a transmission type.

The light emitter 62 is, for example, an LED (Light Emitting Diode) and outputs measurement light. The measurement light is, for example, near infrared light whose wavelength has high biological transmittance. For the measurement light, a wavelength near 805 nanometers equal to an absorption coefficient of oxidized and deoxidized hemoglobin is used.

The light receiver 64 is, for example, a photodiode and receives internal reflected light of the measurement light. The light receiver 64 outputs a measurement signal having a value corresponding to a light reception amount to the pulse wave generator 66. The pulse wave generator 66 generates a volume pulse wave on the basis of the measurement light.

FIG. 4 is a conceptual diagram showing a measurement state of the measurer 6. As shown in FIG. 4, the measurement light of the light emitter 62 scatters in the body of the person to be measured. A part of the scattered light is received by the light receiver 64. An average free stroke of measurement of a wavelength in a near infrared area is approximately one millimeter. Most of the measurement light after incidence on an organism is the scattered light. A measurement value of the scattered light is generally called volume pulse wave.

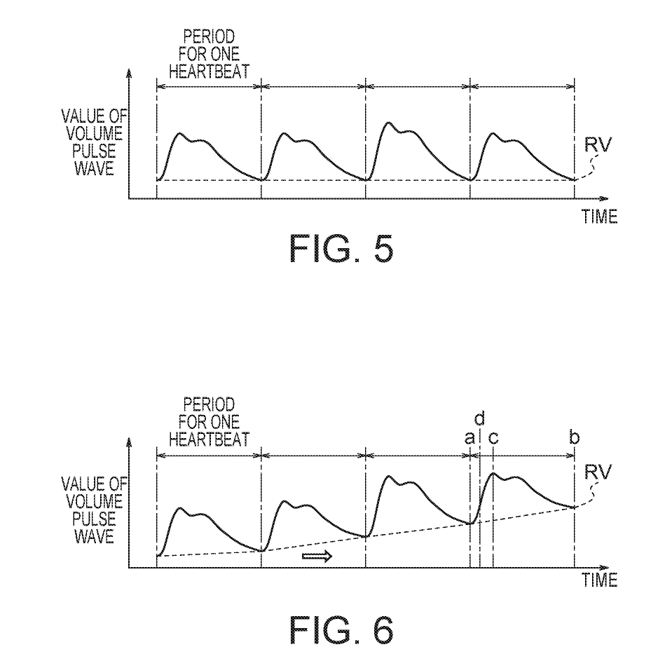

FIG. 5 is a diagram showing an example of the volume pulse wave measured by the measurer 6. The vertical axis indicates a value of the volume pulse wave. The horizontal axis indicates time. The volume pulse wave during rest of the person to be measured is shown in FIG. 5. As shown in FIG. 5, the volume pulse wave repeats fluctuation in every heartbeat. Therefore, the volume pulse wave is formed by a cyclic fluctuation component and a reference level RV. The cyclic fluctuation component is sometimes called AC component or dynamic. The reference level RV is sometimes called DC component, static, offset, or the like. In this embodiment, the reference level RV is set to a value obtained by reducing the fluctuating component from the volume pulse wave. That is, the reference level RV according to this embodiment corresponds to a so-called DC component and is formed by a frequency component having a cycle lower than a cycle of the heartbeat.

FIG. 6 is a diagram showing an example of the volume pulse wave at the time when heat stress is given to the person to be measured. The vertical axis indicates a value of the volume pulse wave. The horizontal axis indicates time. The reference level RV indicates a rising tendency because of the heat stress. This is considered to be because, as heat radiation activity of the person to be measured becomes active, a blood volume in a peripheral blood vessel increases and an absorption amount of the measurement light increases. The blood volume is in a proportional relation with a blood vessel diameter of the peripheral blood vessel. The reference level RV correlates with a light absorption amount at the time when blood is used as an absorber of light. The blood volume and the reference level RV increase because the blood vessel diameter of the person to be measured is expanded by the heat stress. Note that analysis processing for the volume pulse wave in the past focuses on a cyclic fluctuation component having high stability with respect to intensity fluctuation of the measurement light. On the other hand, it is considered difficult to set the reference level RV, a measurement value of which is easily affected by the intensity of the measurement light, as a stable measurement target. In general, the reference level RV has not been used for the analysis processing for the volume pulse wave.

Referring back to FIG. 2, a detailed configuration of the biological-information processing device 12 is explained. The biological-information processing device 12 shown in FIG. 2 performs processing of information based on the volume pulse wave measured by the measurer 6. The biological-information processing device 12 includes an evaluation value acquirer 14, a storage 16, an output unit 18, and a posture detector 20. The biological-information processing device 12 starts the processing of the information based on the volume pulse wave when a measurement start signal is input by a not-shown operation input unit and ends the processing of the information based on the volume pulse wave when a measurement end signal is input by the operation input unit.

The evaluation value acquirer 14 acquires, on the basis of the information concerning the volume pulse wave, an evaluation value indicating the heat radiation activity of the person to be measured. The storage 16 is realized by, for example, a semiconductor memory element such as a RAM (Random Access Memory) or a flash memory, a hard disk, or an optical disk. The storage 16 stores computer programs to be executed by the biological-information processing device 12 and stores information necessary in control processing.

The output unit 18 causes the display 2 and the vibration generator 10 to output the information obtained by the biological-information processing device 12. That is, the output unit 18 includes a display control function for the display 2 and a sound output control function for the vibration generator 10. The posture detector 20 includes, for example, a gyro and outputs physical status information of a subject.

A detailed configuration of the evaluation value acquirer 14 is explained. The evaluation value acquirer 14 shown in FIG. 2 includes a first acquirer 140 and a second acquirer 142.

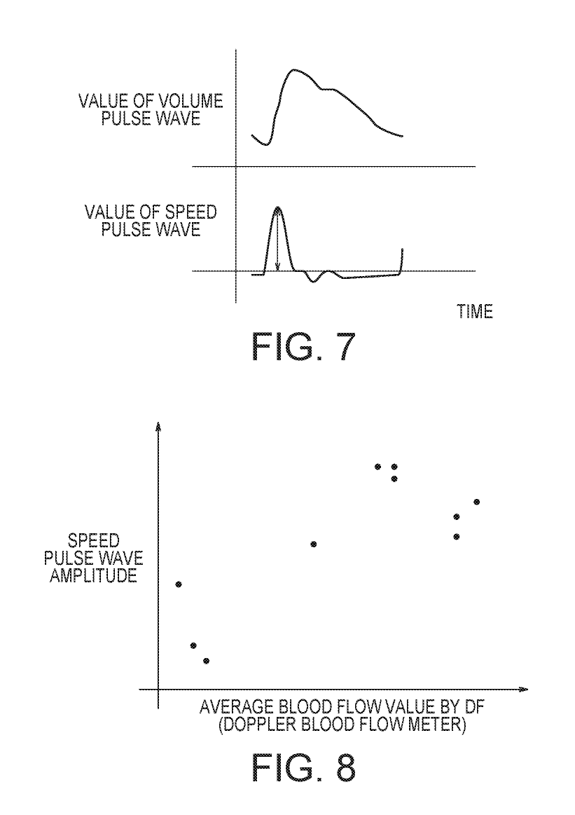

FIG. 7 is a diagram showing a relation between a volume pulse wave and a speed pulse wave for one heartbeat. The upper side of FIG. 7 is a graph showing the volume pulse wave. The vertical axis indicates a value of the volume pulse wave. The lower side of FIG. 7 is a graph showing the speed pulse wave. The vertical axis indicates a value of the speed pulse wave. The horizontal axis indicates time. As shown in FIG. 7, the first acquirer 140 acquires a first index value based on an amplitude value of the speed pulse wave obtained by time-differentiating the volume pulse wave. For example, the first acquirer 140 calculates time differential of the volume pulse wave to acquire the speed pulse wave and acquires a value of the amplitude of the speed pulse wave at each heartbeat (an arrow in FIG. 7). The first index value only has to be a value having a correlation with the amplitude value of the speed pulse wave. The first index value may be, for example, a maximum of the speed pulse wave or a difference value between the maximum and a minimum of the speed pulse wave. The time differential may be performed by differential processing.

FIG. 8 is a diagram showing a relation between the amplitude of the speed pulse wave and an average blood flow value from an arteriole to a venule measured by a Doppler blood flow meter. The vertical axis indicates a value of the amplitude of the speed pulse wave. The horizontal axis indicates the average blood flow value measured by the Doppler blood flow meter. As shown in FIG. 8, the obtained value of the amplitude of the speed pulse wave and a peripheral average blood flow value has a correlation equal to or larger than 0.8. In this way, the obtained first index value has a high correlation with a peripheral blood flow. A value concerning the peripheral blood flow is acquired.

The second acquirer 142 acquires a second index value correlating with the reference level RV of the volume pulse wave at each heartbeat. As shown in FIG. 6, the second acquirer 142 acquires, as the second index value, a value of the volume pulse wave at least at any of the following points in time, for example: a start point in time “a” of a contraction period; an end point in time “b” of an expansion period; and a point in time “c” of a maximum value at each heartbeat. In this way, the second index value is a value at a predetermined point in time at each heartbeat in the volume pulse wave. By acquiring, at each heartbeat, the value of the volume pulse wave at the predetermined point in time such as the start point in time “a” of the contraction period and the end point in time “b” of the expansion period, it is possible to grasp a time-series increasing or decreasing tendency of the reference level RV.

The second acquirer 142 may set, as the second index value, an average of volume pulse waves at the start point in time “a” of the contraction period, the end point in time “b” of the expansion period, and the point in time “c” of the maximum at each heartbeat. The second acquirer 142 may calculate a value at the predetermined point in time at each heartbeat using the average of the volume pulse waves and values of the volume pulse wave at the start point in time “a” of the contraction period, the point in time “c” of the maximum, a point in time “d” when an increase between “a” and “c” is the largest, and the like and set the value as the second index value. These second index values indicate an increasing or decreasing tendency of the reference level RV, that is, a time-series increasing or decreasing tendency of the volume pulse wave and are values concerning a blood vessel diameter.

The second acquirer 142 may acquire the reference level RV with a fluctuation component reduced by filtering processing of the volume pulse wave. For example, the second acquirer 142 performs time average processing of the volume pulse wave to thereby acquire the reference level RV with a fluctuation component due to time reduced.

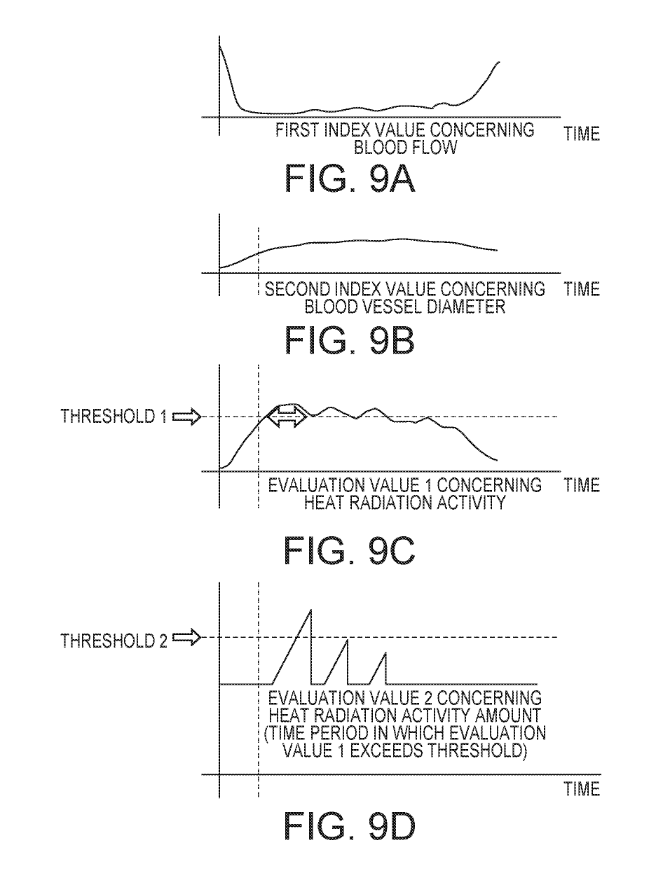

An evaluation value acquired by the evaluation value acquirer 14 is explained more in detail with reference to FIGS. 9A to 9D. FIGS. 9A to 9D are diagrams showing examples of evaluation values. The vertical axis of FIG. 9A indicates a first index value, the vertical axis of FIG. 9B indicates a second index value, the vertical axis of FIG. 9C indicates an evaluation value 1 concerning heat radiation activity, that is, a ratio of the first index value and the second index value at each heartbeat, and FIG. 9D shows an evaluation value 2 concerning the heat radiation activity, that is, a time period in which the evaluation value 1 exceeds a threshold 1. The horizontal axis indicates time.

The evaluation value acquirer 14 acquires, on the basis of the first index value based on the amplitude value of the speed pulse wave and the second index value indicating the time-series increasing or decreasing tendency of the volume pulse wave, an evaluation value indicating an internal state of the person to be measured. More specifically, the evaluation value acquirer 14 acquires, as the evaluation value 1, a value based on a ratio of the first index value and the second index value at each heartbeat. For example, the evaluation value acquirer 14 acquires, as the evaluation value 1, the inverse of a value obtained by dividing the first index value by the second index value at each heartbeat. By calculating the ratio of the first index value and the second index value at each heartbeat in this way, fluctuation in irradiation light intensity of the light emitter 62 can be cancelled. A state of the heat radiation activity of the person to be measured can be more stably evaluated. For example, the evaluation value 1 exceeding a threshold 1 indicates that the heat radiation activity of the person to be measured is activated. The threshold 1 can be set in advance by an evaluation experiment.

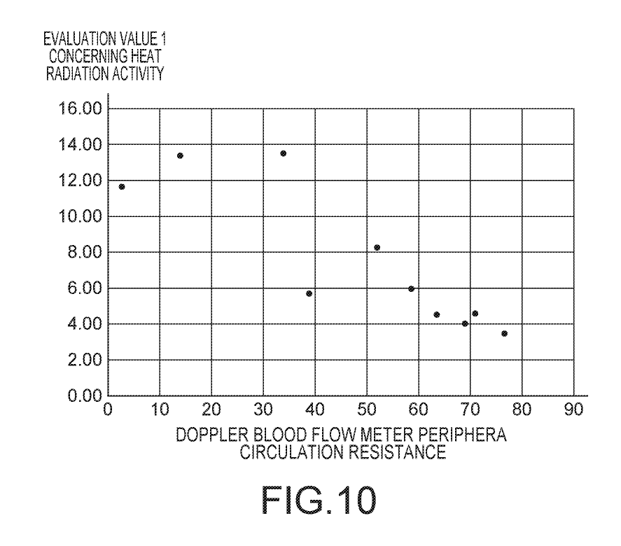

FIG. 10 is a diagram showing a relation between the evaluation value 1 and peripheral circulation resistance measured by the Doppler blood flow meter. The vertical axis indicates the evaluation value 1 and the horizontal axis indicates the peripheral circulation resistance. As shown in FIG. 10, a correlation coefficient between the evaluation value 1 and the peripheral circulation resistance measured by the Doppler blood flow meter is equal to or larger than (−) 0.86. A negative correlation is high. This is because the reference level RV is proportional to a change in a blood volume in a measurement part, that is, a total volume of blood vessels including blood and a fluctuation component is proportional to a blood vessel volume change rate at that time. In this way, the evaluation value 1 has a negative high correlation with the peripheral circulation resistance. Therefore, it is possible to evaluate, with the evaluation value 1, a state of a blood volume increase from an artery to a venule, that is, a state of the heat radiation activity of the person to be measured.

For example, when the evaluation value 1 exceeds the threshold 1, the output unit 18 causes the display 2 and the vibration generator 10 to output notification information indicating that the heat radiation activity of the person to be measured has reached a fixed level. Consequently, the person to be measured can take a preventive action for heatstroke, dehydration, and the like to, for example, reduce an exercise amount and go into the shade. Variation of light intensity of measurement light is cancelled by calculating the ratio of the first index value and the second index value at each heartbeat. A simpler measurement system including an LED or the like can also acquire information concerning peripheral circulation resistance at the same degree of accuracy as the accuracy of the measurement system by the Doppler blood flow meter.

As shown in FIG. 9A to 9D, the evaluation value acquirer 14 acquires, as the evaluation value 2, a time period in which the evaluation value 1 exceeds the threshold 1. That is, the time period in which the evaluation value 1 exceeds the threshold 1 indicates a time period in which a state in which the heat radiation activity of the person to be measured exceeds a fixed level continues. A state in which the evaluation value 1 exceeds the threshold 1 is a state in which the heat radiation activity of the person to be measured continues. Possibility of leading to dehydration and heatstroke increases. For example, when the evaluation value 2 exceeds a threshold 2, the output unit 18 causes the display 2 and the vibration generator 10 to output notification information for urging water intake and rest. By using the evaluation value 2 to urge the person to be measured to drink water and take rest, it is possible to reduce a risk of dehydration and heatstroke.

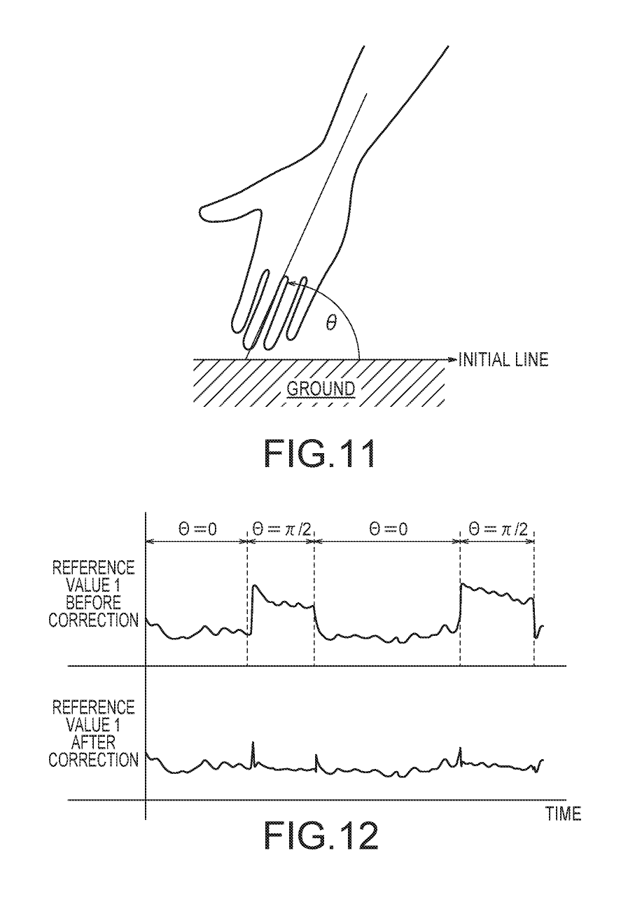

Details of correction processing using the posture detector 20 are explained. FIG. 11 is a diagram showing a radius vector (θ) at the time when the ground is set as an initial line and a fingertip is set as the origin of the initial line. The posture detector 20 outputs a value of the radius vector (θ).

FIG. 12 is a diagram for explaining correction processing by the second acquirer 142. An upper figure shows a reference value 1 before correction at the time when θ is changed to 0, π/2. A lower figure shows the reference value 1 after correction. The vertical axis indicates the reference value 1 after correction. The reference value 1 corresponds to the reference level RV.

The second acquirer 142 corrects the reference level RV according to Equation (1) using a direction θ of the fingertip detected by the posture detector 20. In Equation (1), “u” is an experimentally calculated correction coefficient. For example, u=0.39.

RV=RV×(1+cos θ)÷u Equation (1)

Consequently, the second acquirer 142 can obtain the reference value 1 after correction with the influence on a blood flow by the gravity reduced. It is known that a vein has a low blood pressure and blood circulation is easily affected by the gravity. As shown in the upper figure of FIG. 12, the reference value 1 before correction is conspicuously small at a radius vector of 90°. The reference value 1 before correction depends on the transmittance of measurement light. Therefore, at the radius vector of 90°, a light absorption degree is considered to increase because a blood volume in a measurement part increases according to a change in the radius vector.

Note that the biological-information processing device 12 is made up of, for example, a processor. The word “processor” means, for example, a CPU (Central Processing Unit), a GPU (Graphics Processing Unit), or circuits such as an application specific integrated circuit (ASIC), a programmable logic device (e.g., a simple programmable logic device (SPLD)), a complex programmable logic device (CPLD), and a field programmable gate array (FPGA). The processor realizes a function by reading out and executing a computer program saved in the storage 16. Note that the computer program may be directly incorporated in the circuit of the processor instead of being saved in the storage 16. A plurality of independent processors may be combined to configure the biological-information processing device 12. The processors may realize functions by reading out and executing computer programs.

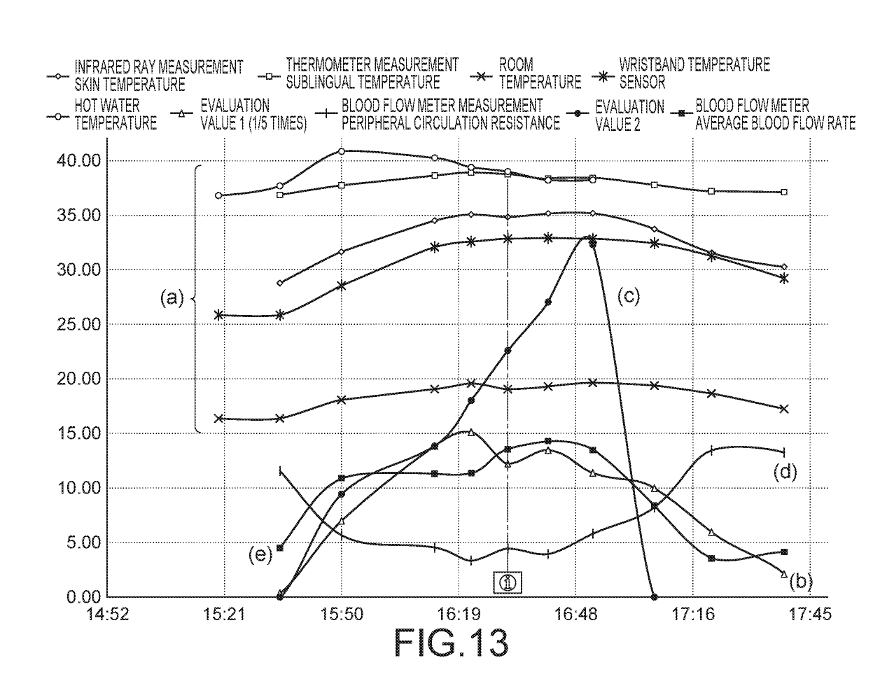

FIG. 13 is a diagram showing a relation between the evaluation values 1 and 2 and a body temperature, an average blood flow rate, and peripheral circulation resistance at the time when a hot heat load is given. In (a), a water temperature, a sublingual temperature, a skin temperature, a wristband temperature sensor, and a room temperature are shown from above. A numerical value of the vertical axis is temperature (° C.) of (a). The wristband temperature sensor is an example of a temperature sensor included in the temperature measurer 4 (FIG. 2). A wristband of the biological measurement device 1 is worn near the forearm wrist of the left arm.

In FIG. 13, (b) indicates the evaluation value 1, (c) indicates the evaluation value 2, (d) indicates the peripheral circulation resistance, (e) indicates the average blood flow rate, and the horizontal axis indicates time. The peripheral circulation resistance of (d) and the average blood flow rate of (e) are values measured by a DF (Doppler blood flow meter).

A period in which the hot heat load is given to the human body is 15:20 to 16:56. The person to be measured receives heat stress of the sublingual temperature exceeding 38.5° C. through heat exposure and sweats much. A heat source is hot water. The periphery of the center of the chest is filled with the heat source. The room temperature is set to 20° C. or less to protect the human body (the head). The threshold 1 is 50 and the threshold 2 is eighteen minutes. At this time, notification of heat stress determination is output by the output unit 18 at 16:31.

The skin temperature is affected by the atmospheric temperature as well and changes to temperatures lower than the sublingual temperature. The measurement value by the temperature sensor included in the body temperature measurer 4 changes by seemingly being affected by the outdoor temperature and the skin temperature of the measurement part. The evaluation value 1 has a negative high correlation with the peripheral circulation resistance. The peripheral circulation resistance is considered to be related to a blood volume increase from an arteriole to a venule and heat radiation from the skin due to the blood volume increase as one of heat radiation mechanisms of the human body. That is, when the peripheral circulation resistance decreases, the blood volume from the arteriole to the venule increases and the heat radiation from the skin also increase. The heat radiation occurs for temperature adjustment and is asynchronous with the heartbeat.

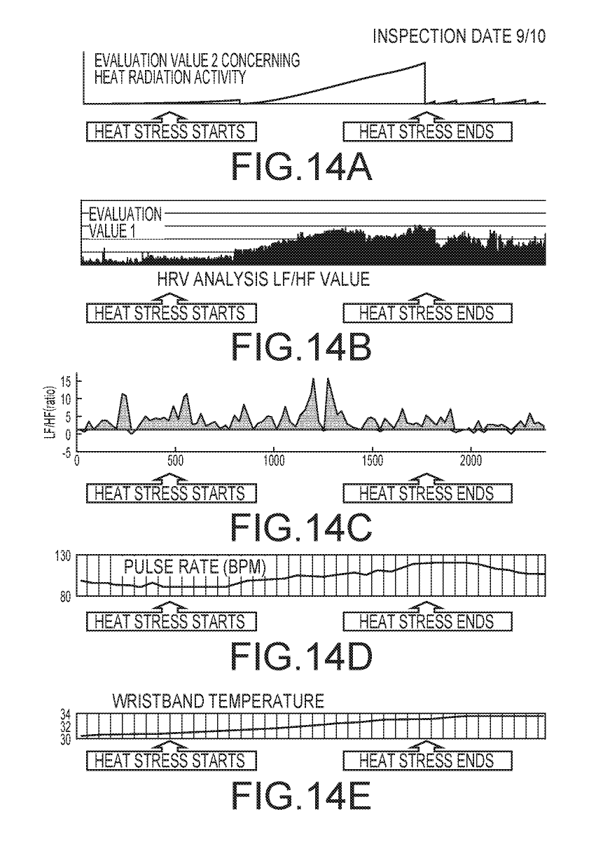

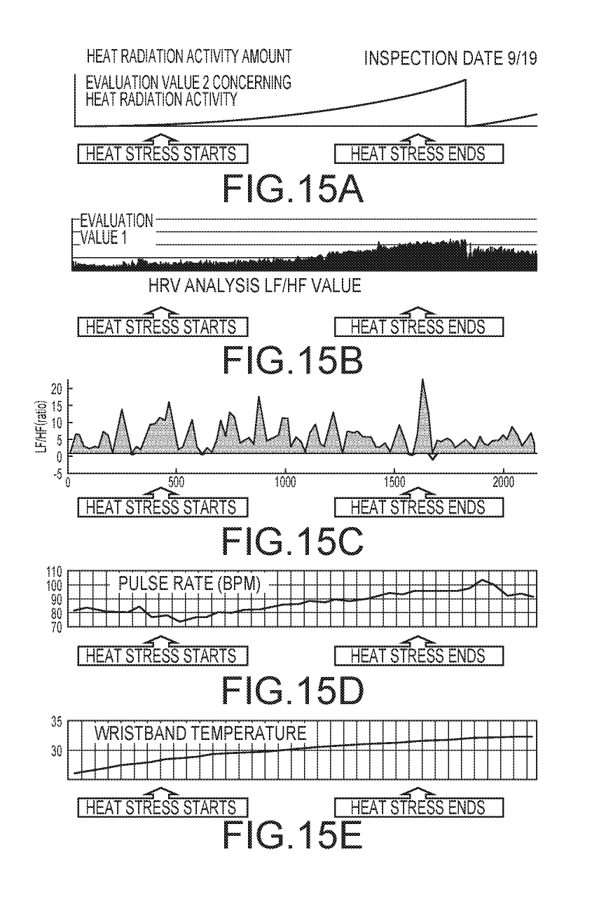

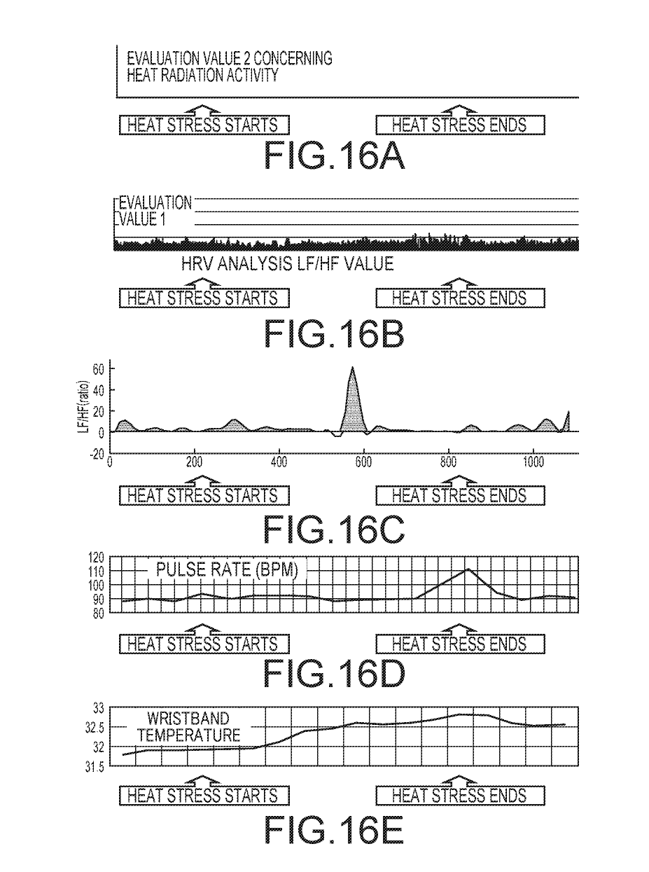

FIGS. 14A to 15E are diagrams showing a relation between the evaluation values 1 and 2 and the body temperature and the HRV analysis at the time when the hot heat load is given. FIGS. 16A to 16E are diagrams showing a relation between the evaluation values 1 and 2 and the body temperature and the HRV analysis at the time when the hot heat load is not given. In the figures, (a) indicates the evaluation value 2, (b) indicates the evaluation value 1, (c) indicates an LF/HF value of the HRV analysis, (d) indicates a pulse rate, (e) indicates a measured temperature by the body temperature measurer 4 (FIG. 2), and the horizontal axis indicates time. The LF/HF value of the HRV analysis is a relation with activity of the autonomic nerve of the person to be measured. The LF/HF value tends to increase as more stress is applied to the person to be measured.

FIGS. 14A to 15E are results of measurement of the same person to be measured on different days. As it is seen from these figures, the evaluation value 2 and the LF/HF value show an increasing tendency when heat stress is applied and show a decreasing tendency when the heat stress is stopped. In this way, the evaluation value 2 has a correlation with a stress state of the person to be measured as well. As it is seen from FIGS. 16A to 16E, when the heat stress is not applied, the evaluation values 1 and 2 do not increase.

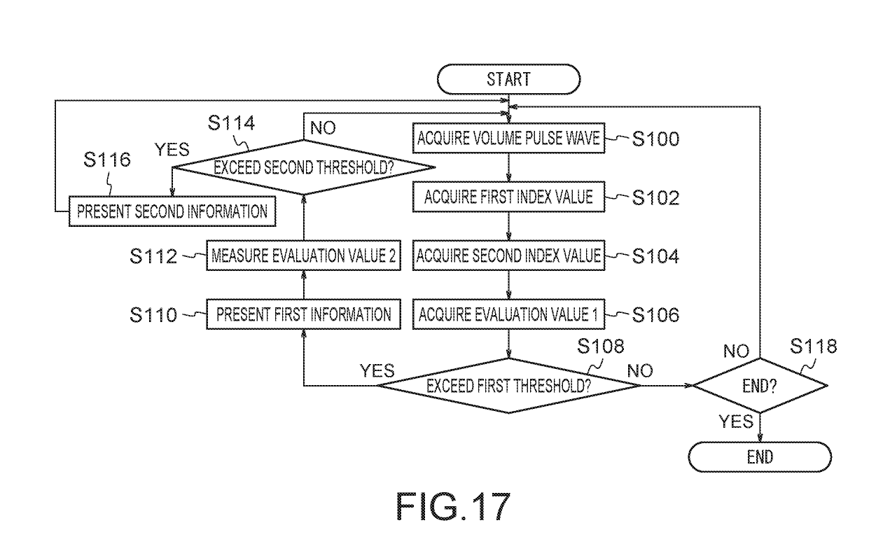

FIG. 17 is a flowchart showing a processing example in which the evaluation values 2 and 3 are used. The measurer 6 performs measurement at each heartbeat and acquires a volume pulse wave (S100). The first acquirer 140 calculates time differential of the volume pulse wave and acquires a first index value (S102). The second acquirer 142 acquires a second index value using the volume pulse wave (S104).

The evaluation value acquirer 14 acquires a ratio of the first index value and the second index value as an evaluation value 1 (S106). Subsequently, the evaluation value acquirer 14 determines whether the evaluation value 1 exceeds a first threshold (S108). When the evaluation value 1 exceeds the first threshold (YES in S108), the evaluation value acquirer 14 causes the output unit 18 to present first information indicating that a thermal activity amount is in an increasing tendency (S110).

Subsequently, when the evaluation value 1 exceeds the first threshold, the evaluation value acquirer 14 measures, as an evaluation value 2, a time period in which the evaluation value 1 exceeds the first threshold (S112). Subsequently, the evaluation value acquirer 14 determines whether the evaluation value 2 exceeds a second threshold (S114). When the evaluation value 2 exceeds the second threshold (YES in S114), the evaluation value acquirer 14 causes the output unit 18 to present second information for urging water intake and rest (S116). On the other hand, when the evaluation value 2 does not exceed the second threshold (NO in S114), the evaluation value acquirer 14 repeats processing from S100.

On the other hand, when the evaluation value 1 does not exceed the first threshold (NO in S108), the evaluation value acquirer 14 determines whether a measurement end signal is input by the operation input unit (S118). When the measurement end signal is not input (NO in S118), the evaluation value acquirer 14 repeats the processing from S100. On the other hand, when the measurement end signal is input (YES in S118), the evaluation value acquirer 14 ends the entire processing.

In this way, the evaluation value acquirer 14 causes, when the evaluation value 1 exceeds the first threshold, the output unit 18 to present information indicating that the thermal activity amount is in the increasing tendency and causes, when the evaluation value 2 exceeds the second threshold, the output unit 18 to present information for urging water intake and rest. Consequently, the person to be measured can objectively grasp a state of thermal activity of the person to be measured and can reduce a risk of heatstroke and dehydration.

As explained above, according to this embodiment, the evaluation value acquirer 14 acquires, on the basis of the first index value based on the amplitude value of the speed pulse wave in the volume pulse wave and the second index value indicating the time-series increasing or decreasing tendency of the volume pulse wave, the evaluation value 1 indicating the internal state of the person to be measured. The evaluation value 1 has the negative high correlation with the peripheral circulation resistance. Therefore, it is possible to indicate, with the evaluation value 1, information concerning a blood volume increase from an artery to a venule, that is, heat radiation activity of the person to be measured.

The evaluation value acquirer 14 sets the ratio of the first index value and the second index value as the evaluation value 1. Therefore, intensity fluctuation of output light of the light emitter 62 can be cancelled. A state of heat radiation activity of the person to be measured can be more stably evaluated.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. A biological measurement device comprising:
a measurer configured to measure a pulse wave;
an evaluation value acquirer configured to acquire, on the basis of a first index value based on an amplitude value of a speed pulse wave obtained by time-differentiating the pulse wave and a second index value correlating with a reference level of the pulse wave at each heartbeat, an evaluation value indicating heat radiation activity; and
an output unit configured to output information corresponding to the evaluation value.

2. The biological measurement device according to claim 1, wherein the evaluation value is a value concerning peripheral circulation resistance.

3. The biological measurement device according to claim 1, wherein the evaluation value is a value based on a ratio of the first index value and the second index value.

4. The biological measurement device according to claim 1, wherein the first index value is a value based on a maximum at each heartbeat of the speed pulse wave.

5. The biological measurement device according to claim 1, wherein the second index value is at least any one of a value at a start point in time of a contraction period at each heartbeat in the pulse wave, a value at a point in time when the speed pulse wave is maximized in an end point in time of the contraction period, and a value based on an average of the pulse wave at each heartbeat.

6. The biological measurement device according to claim 1, wherein, when a time period in which the evaluation value exceeds a first threshold exceeds a second threshold, the output unit outputs notification information concerning the heat radiation activity.

7. The biological measurement device according to claim 1, wherein, when the evaluation value exceeds a first threshold, the output unit outputs information indicating that a thermal activity amount is in an increasing tendency.

8. A biological measurement method comprising:
acquiring a pulse wave; and
calculating, on the basis of a first index value based on an amplitude value of a speed pulse wave obtained by time-differentiating the pulse wave and a second index value correlating with a reference level of the pulse wave at each heartbeat, an evaluation value indicating heat radiation activity.

9. The biological measurement method according to claim 8, wherein the evaluation value is a value concerning peripheral circulation resistance.

10. The biological measurement method according to claim 8, wherein the evaluation value is a value based on a ratio of the first index value and the second index value.

11. The biological measurement method according to claim 8, wherein the first index value is a value based on a maximum at each heartbeat of the speed pulse wave.

12. The biological measurement method according to claim 8, wherein the second index value is at least any one of a value at a start point in time of a contraction period at each heartbeat in the pulse wave, a value at a point in time when the speed pulse wave is maximized in an end point in time of the contraction period, and a value based on an average of the pulse wave at each heartbeat.

13. The biological measurement method according to claim 8, further comprising outputting information corresponding to the evaluation value, wherein
when a time period in which the evaluation value exceeds a first threshold exceeds a second threshold, notification information concerning the heat radiation activity.

14. The biological measurement method according to claim 8, further comprising outputting information corresponding to the evaluation value, wherein
when the evaluation value exceeds a first threshold, information indicating that a thermal activity amount is in an increasing tendency.
